(19) United States
(12) Patent Application Publication
(10) Pub. No.: US 2011/0178050 A1
(43) Pub. Date: Jul. 21, 2011

(54) USE OF CYCLOLIGNANS FOR THE TREATMENT OF A HYPERACTIVE IMMUNE SYSTEM

(75) Inventors: Magnus Axelson, Jarfalla (SE); Olle Larsson, Taby (SE)

(73) Assignee: Axelar AB, Stockholm (SE)

(21) Appl. No.: 13/000,090

(22) PCT Filed: Jun. 18, 2009
(86) PCT No.: PCT/SE09/50772

§ 371 Date: Mar. 24, 2011

Related U.S. Application Data
(60) Provisional application No. 61/074,987, filed on Jun. 23, 2008.

(30) Foreign Application Priority Data
Jun. 23, 2008 (SE) 0801469-8

(51) Int. Cl. A61K 31/357; A61K 31/56; C07D 493/04; A61P 29/00; A61P 1/04; A61P 25/00; A61P 25/28; A61P 11/06; A61P 17/00

(52) U.S. Cl. 514/171; 514/463; 549/432

(57) ABSTRACT

There is disclosed use of certain picro derivatives of cyclolignans for prophylaxis or treatment of diseases or conditions characterised by a hyperactive immune system. Examples of cyclolignans according to the invention include picropodophyllin, deoxypicropodophyllin, anhydropicropodophyllol or deoxyanhydropicropodophyllol. Formula (I)

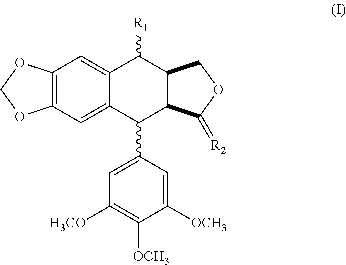

### TECHNICAL FIELD

The present invention relates to certain cyclolignans for prophylaxis and/or treatment of conditions characterized by a hyperactive immune system.

### BACKGROUND

The human immune system has evolved over millions of years to develop sophisticated defence mechanisms to protect us from infecting microbes and their virulence factors. The immune system may be divided into two parts. The first part is the innate, or ancient, immune system, consisting of natural killer (NK) cell lymphocytes, monocytes/macrophages, dendritic cells, neutrophils, basophils, eosinophils, tissue mast cells, and epithelial cells, which recognizes pathogens, e.g. bacteria, and triggers a variety of mechanisms of pathogen elimination. The second part is the adaptive immune system, which is a more recently evolved system of immune responses mediated by T and B lymphocytes. The T cells are thymus-derived lymphocytes that mediate adaptive cellular immune responses, i.e. cell mediated immunity, including helper T, regulatory T, and cytotoxic T lymphocytes. The lymphocytes are bone-marrow derived and express surface immunoglobulin, the B cell receptor for antigen, and secrete specific antibodies after interaction with antigens, making up the humoral immunity. From this, it is evident that lymphocytes play the key roles in the immune system.

The normal immune system has the capacity to distinguish “own” from “foreign” tissues or factors in an organism, but autoimmunity can still occur under some pathological conditions. The typical feature of an autoimmune disease is that tissue injury is caused by an immunologic reaction of the organism with its own tissues. The exact mechanisms behind autoimmunity are not known but contributing factors could be exogenous as well as endogenous. In the latter case, altered antigen presentation, increased T cell help, increased B cell function, apoptotic defects, cytokine imbalance and/or altered immunoregulation, may be involved. Thus, the causes of autoimmune disease can differ and are likely to be multifactorial.

Diseases caused by hyper-reactivity of the immune system are caused by autoimmunity, allergens and transplanted grafts.

The incidence of autoimmune diseases is generally increasing in developed countries and according to one hypothesis this may be a result of an improved way of living, i.e. in an ultra-clean environment there will be very little exposure to exogenous antigens like parasites and bacteria. Many different autoimmune diseases exist, but among the major and/or serious ones are rheumatoid arthritis, Crohn's disease, ulcerative colitis, and multiple sclerosis.

Rheumatoid arthritis is a multisystem disorder in which immunological abnormalities characteristically result in symmetrical joint inflammation, articular erosions and extra-articular complications. It is the most common and disabling autoimmune arthritis and about 1-3% of the Western population are affected. About 10% of these develop severe disease with pain and skeletal and joint deformities.

Crohn's disease is a serious inflammatory bowel disease, which is often chronic with remissions and exacerbations. Common symptoms are diarrhoea, weight loss, abdominal pain (sometimes obstructive), and fever. Local complications include intestinal strictures, perforations, abscesses and fistulas. The patients may need supportive nutritional treatment because of malabsorption.

Ulcerative colitis is also a serious inflammatory bowel disease with relapses and remissions over many years. Features of active disease are frequent diarrhoea with blood and mucus, urgency, sometimes abdominal pain, and loss of weight. The disease increases the risk of developing colonic cancer.

Multiple sclerosis is an inflammatory condition affecting the myelin sheath of CNS but not peripheral neurons. The disease may be benign, follow a relapsing and remitting course, or show an intense progression from the start. The disease can affect the optic nerve, brainstem, cerebellum and spinal cord, the latter resulting in paresis of limbs, bladder and bowel dysfunction etc.

Alzheimer's disease is a neurodegenerative condition, the progression of which appears to decrease by immunosuppressive treatment. The disease is slowly progressing, but eventually leading to dementia, which is characterized by an overall impairment of intellectual, cognitive and memory functions without loss of alertness. About 5% of those over 65 years and 20% of those over 80 years of age have the disease. Although the disease is usually not classified as a regular autoimmune disease, the results of several retrospective studies suggest that anti-inflammatory agents will protect against the dementia (Bird TD and Miller B L, In: Harrison's Principles of internal medicine; Kasper D L et al. eds.; McGraw-Hill, New York, 2005, pp. 2393-2406).

Atopic allergic conditions like asthma and eczematous dermatitis are also common immunological diseases. Eczematous dermatitis (atopic dermatitis) is a relapsing condition usually beginning in infancy and sometimes continuing into adult life. Typical features are intercellular epidermal oedema and pruritus and after becoming chronic lichenification and scaling are seen. Atopy means an inherited tendency to develop an immune hyper-reactivity.

Organ transplantation is an important form of treatment in modern medicine, but a transplanted organ contains antigens that may become targets for the recipient's immune system. Circulating T cells are a major cause of graft (transplant) rejection, but also antibodies toward these alloantigens can mediate transplant rejection. Organ, tissue or cell transplantations, requiring suppression of the immune system, are being performed more and more frequently in humans.

The pathogenesis of autoimmune diseases is not known and many of these diseases lack efficient therapies. Consequently, there is an urgent need for new and effective treatment alternatives for most of these diseases, but also for allergic diseases and after transplantation.

Many of the described diseases lack efficient therapies, although analgesics, nonsteroidal anti-inflammatory drugs (NSAIDs), corticosteroids, disease-modifying drugs and/or immunosuppressive drugs are frequently being used. Consequently there is a need for new and more effective treatment alternatives.

### PRIOR ART

WO 1986/004062 discloses certain cyclolignans as useful against some collagenoses, e.g. rheumatoid arthritis, neurological diseases, e.g. multiple sclerosis, and rejection of transplants. Said cyclolignans are, however, different from those of the present invention.

Specific cyclolignan derivatives have previously been shown to possess immunosuppressive or anti-inflammatory properties in vitro, see Gordaliza M, et al., J Med Chem, 1996, 39, 2865-2868; and Kadota S, et al., Tetrahedron Letters, 1987, 28, 2857-2860.

EP 0711765 A1 also discloses cyclolignan derivatives which are said to have immunosuppressive activity.

WO 2002/102804 discloses the use of certain cyclolignans, including picropodophyllin and deoxypicropodophyllin, for inhibition of the IGF-1 receptor and for treatment of IGF-1R dependent diseases, such as cancer.

WO 2007/097707 discloses the use of certain cyclolignans, including picropodophyllin and deoxypicropodophyllin, for treatment of type 2 diabetes mellitus, macular degeneration and associated diseases, and for contraception.

### SUMMARY OF THE INVENTION

The present invention is based on the observation that certain picro derivatives of cyclolignans (having a cis configuration) can act as immunosuppressive (immunomodulating) agents. Thus, they can be used for the treatment of inflammatory diseases caused by a hyperactive immune system, such as autoimmune diseases, atopic allergy, and graft rejection after transplantation.

In a first aspect there is provided use of a compound of the formula I,

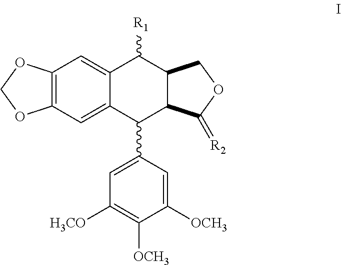

wherein R₁ is selected from the group consisting of H, OH, and an ester group, and wherein R₂ is selected from the group consisting of O and two H, wherein the 5-atom ring has a cis configuration with two beta bonds, as well as a pharmaceutically acceptable salt thereof, for the manufacture of a medicament for prophylaxis or treatment of a hyperactive immune system.

Said compounds of the formula I can be used for the manufacture of a medicament for prophylaxis or treatment of at least one disease selected from the group consisting of rheumatoid arthritis, Crohn's disease, ulcerative colitis, multiple sclerosis, Alzheimer's disease, asthma, eczematous dermatitis, and graft rejection following transplantation.

In a second aspect there is provided a compound of the formula I, as well as a pharmaceutically acceptable salt thereof, for prophylaxis and/or treatment of at least one of said diseases.

In a third aspect there is provided a method of treatment of at least one of said diseases, comprising administering to a subject in need thereof a compound of the formula I, or a pharmaceutically acceptable salt thereof.

In a fourth aspect there is provided use of a compound of the formula I for modulating the immunoreactivity of a mammal.

### DESCRIPTION OF THE DRAWINGS

The invention is described with reference to the drawings in which

FIG. 1 shows the structural formulas of the compounds picropodophyllin and anhydropicropodophyllol; and

FIG. 2 shows the structural formulas of the compounds deoxypicropodophyllin and deoxyanhydropicropodophyllol.

### DETAILED DESCRIPTION

In one aspect the present invention provides use of a compound of the formula I,

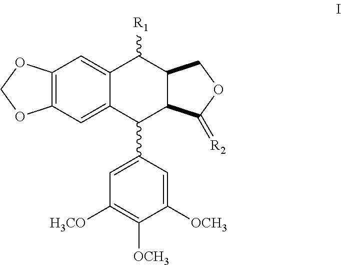

wherein R₁ is selected from the group consisting of H, OH, and an ester group, and R₂ is selected from the group consisting of O and two H, wherein the 5-atom ring has a cis configuration with two beta bonds, as well as a pharmaceutically acceptable salt thereof, for the manufacture of a medicament for prophylaxis or treatment of a hyperactive immune system.

Especially the compounds of the formula I can be used for the manufacture of a medicament for prophylaxis and treatment of at least one disease selected from the group consisting of rheumatoid arthritis, Crohn's disease, ulcerative colitis, multiple sclerosis, Alzheimer's disease, asthma, eczematous dermatitis, and graft rejection following transplantation.

When R₂ is O there is a double bond attaching the oxygen to the carbon of the 5-atom ring as shown in formula I. When R₂ is two hydrogen atoms each hydrogen atom is attached with one bond each to the carbon atom in the 5-atom ring.

Notably, compounds of the formula I are picro derivatives having a 5-atom ring with a cis configuration, i.e. two beta bonds, as indicated by the solid bold lines. R₁ and the trimethoxy-phenyl group can be in either alpha- or beta-position, as is illustrated by wavy lines. The 5-atom ring is shown to the right in formula I.

In one embodiment of the present invention there is provided use of a compound of the formula II,

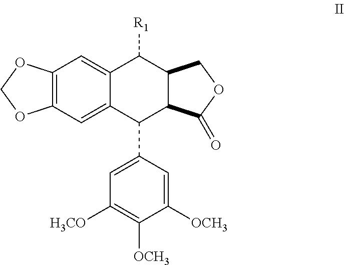

wherein R₁ is selected from the group consisting of H, OH, and an ester group, as well as a pharmaceutically acceptable salt thereof, wherein the lactone ring has a cis configuration with two beta bonds, and wherein R₁ and the trimethoxyphenyl group are in alpha-position, as illustrated by dashed lines. Formula II equals formula I wherein R₂=O and wherein R₁ and the trimethoxyphenyl group are in alpha position. The lactone ring is the five-atom ring shown to the right in formula II.

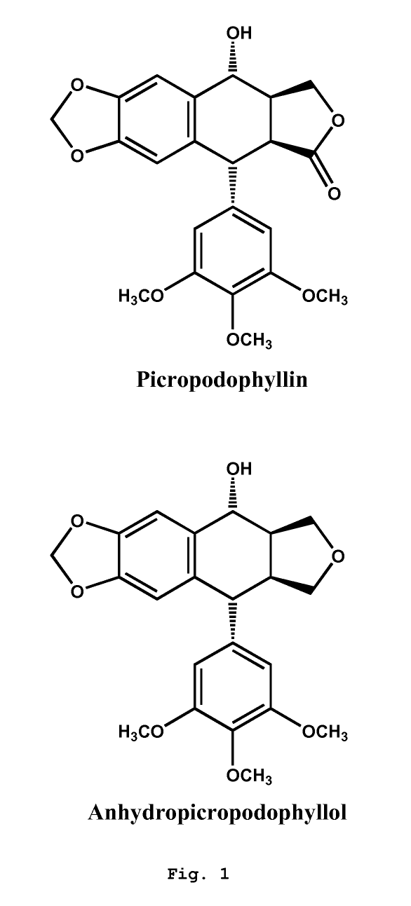

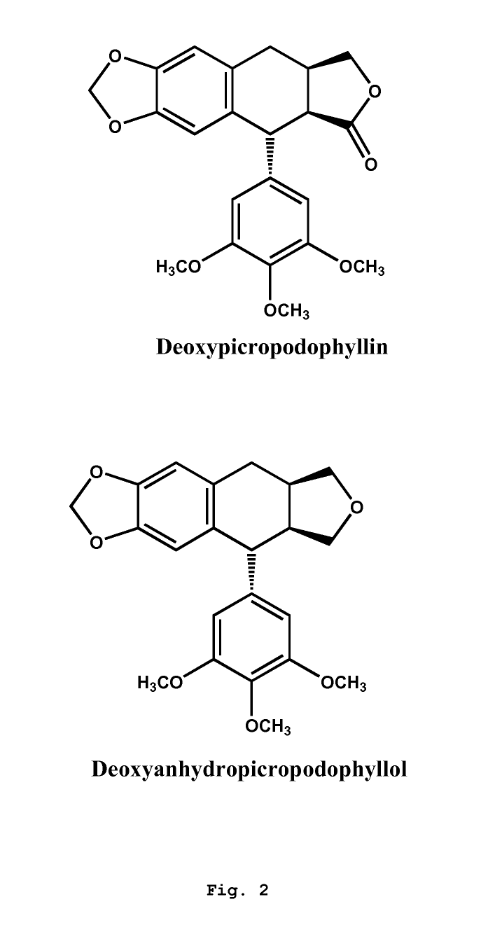

Preferred compounds of the formula II are picropodophyllin (FIG. 1 top) and deoxypicropodophyllin (FIG. 2 top), which compounds have turned out to be particularly suitable for use according to the present invention.

In another embodiment of the present invention there is provided use of a compound of the formula III,

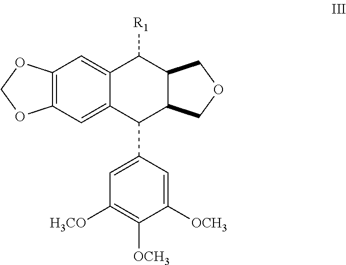

wherein R₁ is selected from the group consisting of H, OH, and an ester group, as well as a pharmaceutically acceptable salt thereof, wherein the cyclo-ether ring has a cis configuration with two beta bonds, indicated by the solid bold lines, and wherein R₁ and the trimethoxyphenyl group are in alpha-position as illustrated by dashed lines. Formula III corresponds to formula I wherein R₂ is two H and wherein R₁ and the trimethoxyphenyl group are in alpha-position.

Preferred compounds of the formula III are anhydropicropodophyllol, that is the picropodophyllin cyclic ether (FIG. 1 bottom) and deoxyanhydropicropodophyllol, that is the deoxypicropodophyllin ether (FIG. 2 bottom).

The use of a mixture of two or more compounds of the formula I for the manufacture of a medicament according to the invention lies within the scope of the invention.

R₁ as an ester group can designate any pharmaceutically acceptable ester group, such as phosphate esters and amino acid esters. The ester group can also comprise a free carboxylic group or another acid group. Especially the ester group can be selected from the group consisting of OCOH, OCO(CH₂)₀₋₁₈CH₃, OCOCH(CH₃)₂, OCO(CH₂)₂COOH, OCOCH₂N(CH₃)₂, OCONHCH₂CH₃, OCOC₅NH₄ and OPO₃H₂. In a particular embodiment R₁ is OCOCH₂N(CH₃)₂. The main purpose of using an ester derivative is to improve the pharmaceutical properties of the compound, for instance the water solubility.

The invention refers to the use of a compound of formula I for the preparation of a medicament for prophylaxis or treatment of different autoimmune diseases. Non-limiting examples of autoimmune diseases that can be prevented or treated by such a compound are rheumatoid arthritis, systemic lupus erythematosus (SLE), Crohn's disease, ulcerative colitis, multiple sclerosis, myasthenia gravis, primary biliary cirrhosis, autoimmune hepatitis, Goodpasture's syndrome, pemphigus vulgaris, type 1 diabetes mellitus, autoimmune gastritis, Addison's disease, pernicious anemia, celiac disease, myositis, Sjögren's syndrome, systemic sclerosis, scleroderma, necrotizing glomerulonephritis, Wegener's granolomatosis, Guillian-Barre's syndrome, Langerhan's histiocytosis, sarcoidosis and autoimmune inflammatory lung diseases. To the group of treatable diseases can also be added Alzheimer's disease.

Furthermore, atopic allergic conditions such as asthma, eczematous dermatitis (atopic dermatitis), rhinitis and urticaria can also be prevented or treated by a compound of the formula I.

Moreover, a compound of the formula I can also be used for the prevention or treatment of graft rejection following transplantation of organs, tissues or cells and of graft-versus-host disease.

There is provided use of any compound mentioned above for the manufacture of a medicament for prophylaxis or treatment of rheumatoid arthritis.

There is provided use of any compound mentioned above for the manufacture of a medicament for prophylaxis or treatment of Crohn's disease.

There is provided use of any compound mentioned above for the manufacture of a medicament for prophylaxis or treatment of ulcerative colitis.

There is provided use of any compound mentioned above for the manufacture of a medicament for prophylaxis or treatment of multiple sclerosis.

There is provided use of any compound mentioned above for the manufacture of a medicament for prophylaxis or treatment of Alzheimer's disease.

There is provided use of any compound mentioned above for the manufacture of a medicament for prophylaxis or treatment of asthma.

There is provided use of any compound mentioned above for the manufacture of a medicament for prophylaxis or treatment of eczematous dermatitis.

There is provided use of any compound mentioned above for the manufacture of a medicament for prophylaxis or treatment of graft rejection following transplantation.

In case of diseases or conditions requiring additional therapy, treatment using the compounds of the invention may be combined with other types of treatments. For example, the compounds can be useful to sensitize cells and potentiate the effect of other treatments. The invention therefore also refers to the use of a compound of the formula I in combination with another type of therapy such as a treatment with a pharmaceutical drug, surgery etc. Examples of drugs or therapies which can be used together with the compounds of the invention for the treatment of autoimmune diseases, allergic conditions or prevention of graft rejection include but are not limited to analgesics (e.g. paracetamol), nonsteroidal anti-inflammatory drugs, NSAIDs (e.g. aspirin, meloxicam), corticosteroids (e.g. prednisolone), disease-modifying drugs (e.g. methotrexate, sulfasalazine) and/or immunosuppressive drugs (e.g. azathioprine, leflunomide, cyclosporine, cyclophosphamide).

There is provided use of any compound mentioned above, including a compound according to formula I, a compound according to formula II and a compound according to formula III, in combination with at least one further drug selected from the group consisting of analgesics (e.g. paracetamol), nonsteroidal anti-inflammatory drugs (NSAIDs, e.g. aspirin, meloxicam), corticosteroids (e.g. prednisolone), disease-modifying drugs (e.g. methotrexate, sulfasalazine) and/or immunosuppressive drugs (e.g. azathioprine, leflunomide, cyclosporine, cyclophosphamide).

In another aspect there is provided a compound according to formula I, as defined above, as well as a pharmaceutically acceptable salt thereof, for prophylaxis and/or treatment of at least one disease selected from the group consisting of rheumatoid arthritis, Crohn's disease, ulcerative colitis, multiple sclerosis, Alzheimer's disease, asthma, eczematous dermatitis, and graft rejection following transplantation.

In still another aspect there is provided a method of treatment of at least one disease selected from the group consisting of rheumatoid arthritis, Crohn's disease, ulcerative colitis, multiple sclerosis, Alzheimer's disease, asthma, eczematous dermatitis, and graft rejection following transplantation, comprising administering to a subject in need thereof a compound according to formula I, as defined above, as well as a pharmaceutically acceptable salt thereof.

In still another aspect there is provided use of a compound of the formula I, as defined above, as well as a pharmaceutically acceptable salt thereof, for modulating the immunoreactivity of a mammal. In a preferred embodiment the mammal is a human.

Podophyllotoxin and deoxypodophyllotoxin, used as starting materials for the syntheses of the described picro derivatives, i.e. cyclolignans with a cis configuration in their lactone or ether ring, are naturally occurring in plants. It is possible to extract the compounds from plants. For the preparation of said substances in pure form, dried and finely ground rhizomes of e.g. Podophyllum emodi or Podophyllum peltatum can be extracted with organic solvents. The extract can subsequently be filtered and concentrated on silica gel. The fractions containing the substances can be collected and the latter further purified by chromatography on acid alumina and silica gel etc., and finally recrystallized.

Picropodophyllin can be prepared from purified podophyllotoxin. For instance podophyllotoxin can be dissolved in 70% aqueous ethanol and to the solution there may be added sodium acetate and the mixture may be refluxed and stirred for a period of time such as 12 h. The mixture can subsequently be cooled and filtered. The precipitated product picropodophyllin can be washed with ethyl acetate, and then purified by recrystallization from absolute ethanol essentially as described by O Buchardt et al. (J Pharmaceut Sci 1986; 75:1076-1080) or purified by chromatography on silica gel, mobile phase: hexane-ethyl acetate mixtures, and/or octadecylsilane-bonded silica, mobile phase: aqueous methanol. The total synthesis of picropodophyllin has been described by J W Gensler et al. (J Am Chem Soc 1960; 82:1714-1727).

Deoxypicropodophyllin can be prepared from purified deoxypodophyllotoxin using essentially the same procedure. A person skilled in the art is able to prepare picropodophyllin and deoxypicropodophyllin in the light of this description and the references which are mentioned in this description.

Anhydropicropodophyllol and deoxyanhydropicropodophyllol can be prepared from picropodophyllin and deoxypicropodophyllin, respectively.

As additional examples of compounds of the formulas II and III can be mentioned various esters of picropodophyllin and anhydropicropodophyllol and pharmaceutically acceptable salts thereof, which can be prepared by conventional procedures.

For oral administration, the compounds of the invention can be formulated into solid or liquid preparations such as capsules, pills, tablets, troches, powders, solutions, suspensions or emulsions.

For topical application the compounds can be administered in the form of an unguent, cream, ointment, lotion, solution or a patch.

For parenteral administration, the compounds may be administered as injectable dosages or by continuous intravenous infusion of a solution, suspension or emulsion of the compound in a physiologically acceptable diluent as the pharmaceutical carrier, which can be a sterile liquid, such as water, alcohols, oils, emulsions, and other acceptable organic solvents, with or without the addition of a surfactant and other pharmaceutically acceptable adjuvants.

For inhalation the compounds may be formulated as powder or solution to be delivered as en aerosol.

The compounds can also be administered in the form of a depot injection or implant preparation, which may be formulated in such a manner as to permit a sustained release of the active ingredient.

It is to be understood that this invention is not limited to the particular embodiments shown here. The following examples are provided for illustrative purposes and are not intended to limit the scope of the invention.

### EXPERIMENTAL

### Materials

### Chemicals

Picropodophyllin (99.5% purity) and deoxypicropodophyllin (99.5% purity) were synthesized from podophyllotoxin (from Sigma and other commercial sources) and deoxypodophyllotoxin (a gift from Analytecon SA, Pre Jorat, Switzerland), respectively. Anhydropicropodophyllol (99% purity) and deoxyanhydropicropodophyllol (99% purity) were synthesized as described below from picropodophyllin and deoxypicropodophyllin, respectively.

### Preparation of Anhydropicropodophyllol

A method for the synthesis of anhydropicropodophyllol giving high yields of the product was developed. Briefly, the tert-butyldimethylsilyl ether of picropodophyllin was first prepared by adding tert-butyldimethylsilyl (t-BDMS) chloride under N₂ to a mixture of picropodophyllin and imidazole in dimethylformamide. The yellow solution was stirred overnight at room temperature and poured into water. The derivative was purified prior to reduction of the lactone group with lithium aluminum hydride in tetrahydrofuran. The latter mixture was then stirred at room temperature for 3 hours yielding the t-BDMS derivative of picropodophyllol having two free hydroxyl groups. To a solution of this compound in dichloromethane was added triphenylphosphine and diethyl azodicarboxylate and the mixture was then stirred at room temperature for 3 hours. The solvent was evaporated and the crude t-BDMS ether of anhydropicropodophyllol was then purified. Underivatized anhydropicropodophyllol was obtained by adding tetrabutyl ammonium fluoride to a solution of the derivative in tetrahydrofuran. The mixture was then stirred at room temperature over night. After purification, pure free anhydropicropodophyllol was obtained as a white solid.

### Preparation of Deoxyanhydropicropodophyllol

Deoxyanhydropicropodophyllol was synthesized from deoxypicropodophyllin in a similar way. Briefly, the lactone group of deoxypicropodophyllin was reduced using lithium aluminum hydride in tetrahydrofuran. The mixture was stirred at room temperature for 3 hours yielding deoxypicropodophyllol having two free hydroxyl groups. To a solution of this compound in dichloromethane were added triphenylphosphine and diethyl azodicarboxylate and the mixture was then stirred at room temperature for 3 hours. The pure product deoxyanhydropicropodophyllol was then obtained after purification.

### Experiments

All in vivo experiments were performed according to the ethical guidelines for laboratory animal use and were approved by the local ethics committee.

### Experiment 1. Toxic Effects of Trans and Cis Isomers of Cyclolignans in Mice

The experiment was carried out to determine the significance of the stereochemistry of cyclolignans for their general toxicity by investigating systemic toxic effects of trans and cis isomers of cyclolignans in mice. Five-week old pathogen-free nude mice (nu/nu) were used and housed within plastic isolators in a sterile facility. Experimental treatments with podophyllotoxin, deoxypodophyllotoxin, picropodophyllin and deoxypicropodophyllin were performed by injecting each compound dissolved in 10 microL of a mixture of DMSO and saline intraperitoneally daily for 5 days. The dose injected once daily was 28 mg/kg/d and 6 mice were used for each drug. Control mice were treated with the vehicle only.

The mice were checked daily for signs of discomfort, diseases and weight loss. The results are shown in Table 1. The mice treated with podophyllotoxin became quickly sick and on the second day all of the animals were dead. Deoxypodophyllotoxin-treated mice exhibited serious toxic signs on the second day and after 3 days they were all dead. In contrast, mice treated with either picropodophyllin or deoxypicropodophyllin survived the whole 5-day period and did not exhibit any obvious toxic signs (Table 1). The results show that the cyclolignans picropodophyllin and deoxypicropodophyllin, having a lactone ring with cis configuration are much less toxic than their corresponding trans isomers podophyllotoxin and deoxypodophyllotoxin. The latter two are known to be microtubule inhibitors and this may explain their toxicity.

TABLE 1
Systemic toxicity of trans and cis isomers of cyclolignans in mice

No. of surviving mice/
No. of treated mice
Day

| Compound and dose in mg/kg/d | 1 | 2 | 3 | 4 | 5 |
|---|---|---|---|---|---|
| Solvent | 6/6 | 6/6 | 6/6 | 6/6 | 6/6 |
| Podophyllotoxin, 28 mg | 2/6 | 0/6 | 0/6 | 0/6 | 0/6 |
| Deoxypodophyllotoxin, 28 mg | 6/6 | 4/6 | 0/6 | 0/6 | 0/6 |
| Picropodophyllin, 28 mg | 6/6 | 6/6 | 6/6 | 6/6 | 6/6 |
| Deoxypicropodophyllin, 28 mg | 6/6 | 6/6 | 6/6 | 6/6 | 6/6 |

The results demonstrate that the configuration of the lactone ring in cyclolignans is of crucial importance for their toxicity, i.e. podophyllotoxin and deoxypodophyllotoxin, having a lactone ring with trans configuration, are generally toxic in contrast to their corresponding cis isomers picropodophyllin and deoxypicropodophyllin.

### Experiment 2. Effect of Picropodophyllin on Blood Lymphocytes in Mice

In this experiment, healthy 20-gram mice were treated with picropodophyllin by intraperitoneal injections twice daily, the dose being 20 mg/kg/12 h using DMSO/sunflower oil, 9:1, as vehicle. The control group was treated with the vehicle only (totally per day: 20 μL DMSO/oil). Each group included 3 mice. After treatment for 7 days, the mice were sacrificed and blood samples were taken. The number of blood cells in the samples was counted using an Advia 120 Hematology system from Bayer AB (Diagnostica). The results are shown in Table 2.

TABLE 2
Effect of a seven-day treatment with picropodophyllin (PPP) on blood
lymphocytes in mice. The mean and standard deviation (SD) were used
as measures of central tendency and variation, respectively.

| | Lymphocytes in blood |
|---|---|
| Mice | (×10⁸/L) |
| Controls | |
| No. 1 | 1.9 |
| No. 2 | 3.5 |
| No. 3 | 3.5 |
| PPP-treated | |
| No. 1 | 0.3 |
| No. 2 | 0.1 |
| No. 3 | 0.0 |
| Controls | 2.97 SD: 0.92 |
| PPP-treated | 0.13 SD: 0.15 |

The results show that treatment with picropodophyllin reduced the number of blood lymphocytes in mice, consistent with a suppressive effect on their immune system.

### Experiment 3. Effect of Picropodophyllin on Blood Lymphocytes in Rats

In this experiment, healthy 200-grams rats (Sprague-Dawley) were treated with picropodophyllin by intraperitoneal injections twice daily, the dose being 20 mg/kg/12 h, using DMSO/sunflower oil, 9:1, as vehicle. The control group was treated with the vehicle only (totally per day: 200 μL DMSO). Each group included 5 rats. After treatment for 14 days, the rats were sacrificed and blood samples were taken. The number of blood cells in the samples was counted using an Advia 120 Hematology system from Bayer AB (Diagnostica). The results are shown in Table 3.

TABLE 3
Effect of a two-week treatment with picropodophyllin (PPP) on blood
lymphocytes in rats. The mean and standard deviation (SD) were used
as measures of central tendency and variation, respectively.

| | Lymphocytes in blood |
|---|---|
| Rats | (×10⁹/L) |
| Controls | |
| No. 1 | 10.08 |
| No. 2 | 2.13 |
| No. 3 | 7.41 |
| No. 4 | 4.96 |
| No. 5 | 7.75 |
| PPP-treated | |
| No. 1 | 3.16 |
| No. 2 | 0.45 |
| No. 3 | 4.20 |
| No. 4 | 2.80 |
| No. 5 | 3.86 |
| Controls | 6.48 SD: 3.04 |
| PPP-treated | 2.89 SD: 1.46 |

The results show that treatment with picropodophyllin decreased the blood lymphocytes in rats by more than 50%, consistent with a suppressive effect on their immune system.

### Experiment 4. Effects of Picropodophyllin and Deoxypicropodophyllin on the Activation of Helper T Lymphocytes and Cytotoxic T Lymphocytes in Human Blood

The effects of picropodophyllin and deoxypicropodophyllin on the activation of subgroups of lymphocytes in human blood were determined by flow cytometry (FACS). Lymphocytes from human peripheral blood were purified by ficoll-hypaque (Pharmacia, Amersham) and then distributed in FACS tubes at 500,000 cells in 0.5 mL per tube. Cells were kept in RPMI medium (Life technologies) supplemented with 10% bovine serum for activation of helper T and cytotoxic T cells (BGS; Sigma), 1% penicillin-streptomycin (Sigma) and 1% glutamine (Sigma). The cells were incubated for 30 min at room temperature in the absence (control lymphocytes) or presence of picropodophyllin and deoxypicropodophyllin at 0.1 and 2.5 microM concentrations. After washing, the cells were stained 15 min for the surface molecules CD4, CD8 and CD69. CD4 and CD8 are expressed by two subsets of T lymphocytes, i.e. helper T cells and cytotoxic T cells, respectively, and CD69 is a lymphocyte activation marker. The staining was performed with the following fluorophore conjugated antibodies: anti-CD4PB; anti-CD8APC; anti-CD69APCCy7 (Beckton Dickinson). The cells were then washed with PBS containing 2% FCS and 0.05% NaN3 (FACS buffer) and then stained with propyl iodide (PI)/FITC Annexin V (Beckton Dickinson) to determine apoptosis of cells, and after incubation for another 15 min and washing, the cells were analysed by flow cytometry (FACSCalibur; Beckton Dickinson). Data were evaluated using the Cellquest computer software (Beckton Dickinson). The results are shown in Table 4.

TABLE 4
Effects of picropodophyllin and deoxypicropodophyllin on
the activation of helper T lymphocytes and cytotoxic T lymphocytes
in human blood. All samples were in triplicate.

| | | PPP | PPP | DPPP | DPPP |
|---|---|---|---|---|---|
| | Control | 0.1 μM | 2.5 μM | 0.1 μM | 2.5 μM |
| CD4* | 36.6% | 36.7% | 36.3% | 36.7% | 37.1% |
| CD4/CD69** | 9.2% | 11.0% | 9.7% | 13.2% | 7.5% |
| CD8* | 35.6% | 35.0% | 34.8% | 34.7% | 32.8% |
| CD8/CD69** | 5.8% | 3.2% | 1.8% | 4.1% | 0.9% |
| PI* | 0% | 0% | 0% | 0% | 0% |
| ANNEXIN V* | 1.3% | 0.9% | 0.9% | 1.3% | 1.5% |
| PI | | | | | |
| ANNEXIN V* | 7.2% | 9.4% | 10.5% | 9.2% | 9.3% |
| Total | 6648 | 8749 | 9799 | 8362 | 8361 |
| cells | | | | | |

*Percent helper T cells and cytotoxic T cells of total cells (events).
**Percent activated helper T cells of total helper T cells and percent activated cytotoxic T cells of total cytotoxic T cells.

In the untreated control about 9% of the helper T cells (CD4/CD69) and about 6% of the cytotoxic T cells (CD8/CD69) were activated. Both picropodophyllin and deoxypicropodophyllin dose-dependently decreased the number of activated cytotoxic T lymphocytes (CD8/CD69). These results are consistent with significant immunosuppressive effects of the cyclolignans. The cyclolignans did not induce apoptosis of the cells as shown by the PI and Annexin analyses.

The results described here suggest effects of the cyclolignans on the autoimmune diseases rheumatoid arthritis, Crohn's disease, ulcerative colitis and multiple sclerosis, as well as on graft rejection reactions following transplantation and allergic diseases in a model system. They may also be a model for Alzheimer's disease.

### Experiment 5. Effects of Picropodophyllin and Deoxypicropodophyllin on Proliferation of Lymphocytes in Human Blood

The effects of picropodophyllin and deoxypicropodophyllin on human T lymphocyte function, i.e. the proliferation capacity of human T lymphocytes in response to an antigen/mitogen, were measured using a time course tritium labelled thymidine incorporation assay. Lymphocytes from peripheral blood of patients were purified by ficoll-hypaque (Pharmacia, Amersham) and were distributed in 96 well plates at 100,000 cells per well, and all samples were in triplicates. Cells were kept in RPMI 1640 medium (Life technologies) supplemented with 10% human serum (Sigma), 100 units/mL penicillin (Sigma), 100 microg/mL streptomycin (Sigma) and 2 mM L-glutamine (Sigma). Proliferation of lymphocytes was stimulated with the T lymphocyte mitogen Concanavalin A (Con A) 10 microg/mL (Sigma). After three days incubation with Con A, with and without the cyclolignans, 1 microCi ³H-thymidine (Amersham) was added to each well, and after another eighteen hours cells were harvested and washed, and the amount of ³H-thymidine incorporated into newly synthesized DNA determined by scintillation counting. The results are shown in Table 5.

TABLE 5
Incorporation of ³H-thymidine into T lymphocytes during 18 h after
stimulation with T cell mitogen Concanavalin A for three days in
the absence (control cells) or presence of picropodophyllin (PPP;
2.5 microM) or deoxypicropodophyllin (DPPP; 2.5 microM).

³H-thymidine in cyclolignan-
treated lymphocytes
(% of ³H-thymidine in control
lymphocytes)

| Disease | PPP-treated | DPPP-treated |
|---|---|---|
| RA* | 15 | 7 |
| CD* | 8 | 8 |
| UC* | 13 | 10 |
| MS* | 10 | 8 |
| AD* | 7 | 9 |

*RA = rheumatoid arthritis; CD = Crohn's disease; UC = ulcerative colitis; MS = multiple sclerosis; AD = Alzheimer's disease.

The results show that proliferation of peripheral blood T lymphocytes was suppressed by picropodophyllin and deoxypicropodophyllin in autoimmune diseases.

1. A method of manufacturing a medicine, the method comprising:
combining a compound of formula I, 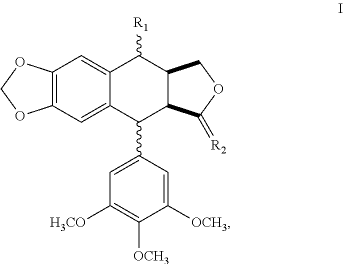
wherein
R₁ is selected from the group consisting of H, OH, and an ester group,
R₂ is selected from the group consisting of O and two H, and
the 5-atom ring fixed to the cyclohexyl ring has a cis configuration with two beta bonds,
or a pharmaceutically acceptable salt of the compound,
with a physiologically acceptable carrier.

2. The method of claim 1, wherein
R₂=O, and
R₁ and the trimethoxyphenyl group are in alpha-position.

3. The method of claim 1, wherein
R₂ is two H, and
R₁ and the trimethoxyphenyl group are in alpha-position.

4. The method of claim 1, wherein R₁ is selected from the group consisting of OCOH, OCO(CH₂)₀₋₁₈CH₃, OCOCH(CH₃)₂, OCO(CH₂)₂COOH, OCOCH₂N(CH₃)₂, and OPO₃H₂.

5. The method of claim 1, wherein the compound is selected from the group consisting of picropodophyllin and deoxypicropodophyllin.

6. The method of claim 1, wherein the compound is selected from the group consisting of anhydropicropodophyllol and deoxyanhydropicropodophyllol.

7. The method of claim 1, further comprising combining at least one further drug selected from the group consisting of an analgesic, a nonsteroidal anti-inflammatory drug (NSAID), a corticosteroid, a disease-modifying drug, and an immunosuppressive drug,
with the compound and/or the salt of the compound and/or the carrier.

8. A compound according to formula I, 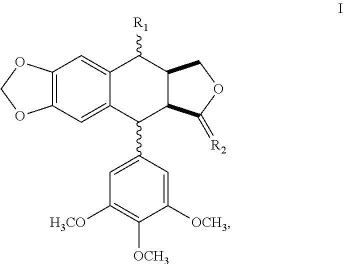
wherein
R₁ is selected from the group consisting of H, OH, and an ester group,
R₂ is selected from the group consisting of O and two H, and
the 5-atom ring fused to the cyclohexyl group has a cis configuration with two beta bonds,
or a pharmaceutically acceptable salt of the compound.

9. A method of treatment of at least one disease selected from the group consisting of rheumatoid arthritis, Crohn's disease, ulcerative colitis, multiple sclerosis, Alzheimer's disease, asthma, and eczematous dermatitis, comprising administering to a subject in need thereof an effective dose of a compound according to formula I, 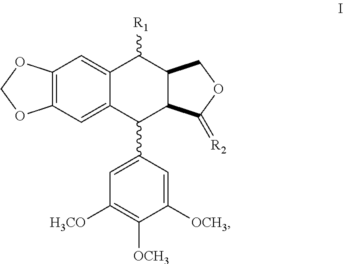
wherein
R₁ is selected from the group consisting of H, OH, and an ester group,
R₂ is selected from the group consisting of O and two H, and
the 5-atom ring fused to the cyclohexyl group has a cis configuration with two beta bonds,
or a pharmaceutically acceptable salt of the compound.

10. The method of claim 2, wherein R₁ is selected from the group consisting of OCOH, OCO(CH₂)₀₋₁₈CH₃, OCOCH(CH₃)₂, OCO(CH₂)₂COOH, OCOCH₂N(CH₃)₂, and OPO₃H₂.

11. The method of claim 3, wherein R₁ is selected from the group consisting of OCOH, OCO(CH₂)₀₋₁₈CH₃, OCOCH(CH₃)₂, OCO(CH₂)₂COOH, OCOCH₂N(CH₃)₂, and OPO₃H₂.

12. The method of claim 2, wherein the compound is selected from the group consisting of picropodophyllin and deoxypicropodophyllin.

13. The method of claim 3, wherein the compound is selected from the group consisting of anhydropicropodophyllol and deoxyanhydropicropodophyllol.

14. The method of claim 2, further comprising combining
at least one further drug selected from the group consisting of an analgesic, a nonsteroidal anti-inflammatory drug (NSAID), a corticosteroid, a disease-modifying drug, and an immunosuppressive drug,
with the compound and/or the salt of the compound and/or the carrier.

15. The method of claim 3, further comprising combining
at least one further drug selected from the group consisting of an analgesic, a nonsteroidal anti-inflammatory drug (NSAID), a corticosteroid, a disease-modifying drug, and an immunosuppressive drug,
with the compound and/or the salt of the compound and/or the carrier.

16. The method of claim 4, further comprising combining
at least one further drug selected from the group consisting of an analgesic, a nonsteroidal anti-inflammatory drug (NSAID), a corticosteroid, a disease-modifying drug, and an immunosuppressive drug,
with the compound and/or the salt of the compound and/or the carrier.

17. The method of claim 5, further comprising combining
at least one further drug selected from the group consisting of an analgesic, a nonsteroidal anti-inflammatory drug (NSAID), a corticosteroid, a disease-modifying drug, and an immunosuppressive drug,
with the compound and/or the salt of the compound and/or the carrier.

18. The method of claim 6, further comprising combining
at least one further drug selected from the group consisting of an analgesic, a nonsteroidal anti-inflammatory drug (NSAID), a corticosteroid, a disease-modifying drug, and an immunosuppressive drug,
with the compound and/or the salt of the compound and/or the carrier.

19. The method of claim 10, further comprising combining
at least one further drug selected from the group consisting of an analgesic, a nonsteroidal anti-inflammatory drug (NSAID), a corticosteroid, a disease-modifying drug, and an immunosuppressive drug,
with the compound and/or the salt of the compound and/or the carrier.

20. The method of claim 11, further comprising combining
at least one further drug selected from the group consisting of an analgesic, a nonsteroidal anti-inflammatory drug (NSAID), a corticosteroid, a disease-modifying drug, and an immunosuppressive drug,
with the compound and/or the salt of the compound and/or the carrier.
